(19) United States
(12) Patent Application Publication
(10) Pub. No.: US 2010/0048936 A1
(43) Pub. Date: Feb. 25, 2010

(54) PROCESS FOR THE PREPARATION OF NITROGEN-CONTAINING COMPOUNDS

(76) Inventors: Alexander Volker A.V. Peters, Senden (DE); Bruce Gordon B.G. Anderson, Vanderbijlpark (ZA); Robert R. Pestman, Eindhoven (NL); Marijke Hilde Leen M.H.L. Groothaert, Maasmechelen (BE); Tjay Tjien T.T. Tjioe, Sittard (NL); Ramesh R. Kanaparthi, Maysprings (SG)

Correspondence Address: NIXON & VANDERHYE, PC, 901 NORTH GLEBE ROAD, 11TH FLOOR, ARLINGTON, VA, 22203, US

(21) Appl. No.: 12/306,684

(22) PCT Filed: Jun. 27, 2007
(86) PCT No.: PCT/EP07/05497

§ 371 Date: Aug. 17, 2009

(30) Foreign Application Priority Data
Jun. 27, 2006 (EP) 06013186.9

(51) Int. Cl. C07C 255/01

(52) U.S. Cl. 558/319

(57) ABSTRACT

Process for the preparation of a nitrogen-containing compound, comprising the steps of: a) bringing N₂, optionally NH₃ and optionally a recycle stream together with a carbon- and hydrogen-containing compound or a carbon-containing compound and H₂ to form a reaction mixture, whereby the ammonia in the reaction mixture, if present, originates for at least 30 wt. % from the recycle stream; b) bringing the reaction mixture in contact with a catalyst at a temperature lying between 200° C. and 800° C. and at a space velocity lying between 10² and 10⁶ ml/(g.h), said catalyst containing a metal M₁ on a support, M₁ being chosen from the group consisting of metals in group 3, 4, 5, 6, 7, 8, 9, 10, 11 and 12 of the IUPAC Periodic Table of Elements or mixtures thereof, whereby the nitrogen-containing compound is formed; c) optionally separating, subsequent to step b), a portion of between 1 and 99 vol. % off from the reaction mixture, said portion being the recycle stream.

The invention relates to a process for the preparation of nitrogen-containing compounds, i.e. compounds whose overall formula comprises CₓH_{y}N_{z} whereby neither one of x, y, or z is zero.

Such a process is known as for example the well-known Andrussow process for the production of hydrogen cyanide (HCN), as referred to on for example page 44-45 of ‘Industrial Organic Chemistry’ by K. Weissermel, H. -J. Arpe; 3^{rd} edition, 1997, VCH Verlag, ISBN 3-527-28838-4 Gb. In the known Andrussow process, HCN is produced. In principle, it is an ammoxidation of methane:

$${CH}_{4} + {NH}_{3} + 1.5 {O}_{2} {\to }^{catalyst} HCN + 3 {H}_{2} O$$

The catalyst is usually platinum, either as a gauze or on a support, with additives such as rhodium. The reaction takes place at atmospheric pressure and 1000-1200° C. with a very short residence time. The reaction gas is rapidly quenched in order to avoid decomposition of HCN. After an acid wash, pure HCN is obtained by distillation from the diluted aqueous solution.

The known process has as disadvantage that it starts from raw materials comprising NH₃. NH₃ is a compound that itself needs to be synthesised, thereby adding to the complexity and cost of obtaining the nitrogen-containing compound.

It is the objective of the present invention to reduce or even eliminate the said disadvantage.

The objective is achieved in that the process comprises the steps of:
a) bringing N₂, optionally NH₃ and optionally a recycle stream together with a carbon- and hydrogen-containing compound or a carbon-containing compound and H₂ to form a reaction mixture, whereby the ammonia in the reaction mixture, if present, originates for at least 30 wt. % from the recycle stream;
b) bringing the reaction mixture in contact with a catalyst at a temperature lying between 200° C. and 800° C. and with a space velocity lying between 10² and 10⁶ ml/g.h, said catalyst containing a metal M₁ on a support, M₁ being chosen from the group consisting of metals in group 3, 4, 5, 6, 7, 8, 9, 10, 11 and 12 of the IUPAC Periodic Table of Elements or mixtures thereof, whereby the nitrogen-containing compound is formed;
c) optionally separating, subsequent to step b), a portion of between 1 and 99 vol. % off from the reaction mixture, said portion being the recycle stream.

The advantage of the process according to the invention is that nitrogen-containing compound can be prepared in fewer steps than was hitherto known, whereby the amount of NH₃ to be used as raw material is reduced or even eliminated.

The process according to the invention relates to a process for the preparation of a nitrogen-containing compound. The term nitrogen-containing compound is understood to mean a compound whose overall chemical formula comprises Carbon, Hydrogen and Nitrogen; this may be expressed by stating that the overall chemical formula comprises CₓH_{y}N_{z} whereby neither one of x, y, or z is zero. Other elements such as oxygen and/or others may also be present in the overall chemical formula of the nitrogen-containing compound. Nitrogen-containing compounds as such are very well-known; examples of nitrogen-containing compounds are hydrogen cyanide (HCN), dimethylamine ((CH₃)₂NH), cyanamide (H₂NCN), dicyandiamide (C₂H₄N₄), urea (NH₂CONH₂), melamine (C₃H₆N₆) and cyanic acid (HOCN).

The process according to the invention comprises the step a) of forming a reaction mixture. The reaction mixture can be formed by bringing N₂ together with a carbon- and hydrogen-containing compound. Carbon- and hydrogen-containing compounds are as such known; a preferred example of such a compound is methane (CH₄). Optionally, it is possible to add hydrogen (H₂) to the reaction mixture, even when already a carbon- and hydrogen-containing compound is used to form the reaction mixture.

The reaction mixture may alternatively also be formed by bringing N₂ together with a carbon-containing compound and hydrogen. Carbon-containing compounds are as such known; examples of such compounds are carbon itself or carbon monoxide.

In the process according to the invention, N₂ is a primary source of nitrogen for forming the nitrogen-containing compound. Since it is an advantage of the process according to the invention that a nitrogen-containing compound is formed in only a few steps, counting from the cheapest and most readily available raw materials, it may be less advantageous to bring more complicated nitrogen-containing raw materials than nitrogen gas such as ammonia or nitrous oxides such as NO or other NOₓ compounds into the reaction mixture. It is thus preferred that the amount of nitrogen-containing raw materials not being N₂ represents at most 50 wt. %—calculated on nitrogen itself—of the total amount of nitrogen in the reaction mixture. At the same time it should be observed that if ammonia is present, the amount of ammonia in the reaction mixture as it is being formed in step a) and as it is being fed into the next step b) is for at least 30 wt. %—calculated on the total amount of ammonia that is being fed to step b)—originating from the recycle step c) to be discussed below. More preferably, the amount of nitrogen-containing raw materials not being N₂ represents at most 40, 30, 20, 10, 5, or even at most 2 wt. %. Most preferably, the amount of nitrogen-containing raw materials not being N₂ is essentially zero.

The terms ‘essentially’, ‘consist essentially of’, ‘constitute essentially all’ or equivalents have the usual meaning that no other compounds or measures are present or taken that have significant impact on the working, effects or achieved objectives of the invention.

In a preferred embodiment of the invention a recycle stream is brought together with the abovementioned raw materials. The recycle stream is a portion of the reaction mixture that is separated off from the reaction mixture after the reaction step b) has been performed at least partly. The advantage of working with a recycle stream as disclosed here is that the process may be steered towards desirable partial or subsequent reactions or increased conversion of the raw materials.

In a further preferred embodiment of the invention a stream containing O₂ and/or an oxygen-containing compound is additionally used to form the reaction mixture. This has the advantage that the occurrence of certain oxidation reactions or partial oxidation reactions can be enhanced; the resulting partially or wholly oxidized compounds may for example constitute useful intermediate compounds in obtaining nitrogen-containing compounds. Examples of oxygen-containing compounds that may be used are CO and H₂O; nitrous oxides may also be used although the limits on the use of nitrous oxides as raw material as given above should be respected. The amount of O₂ and/or another oxygen-containing compound as present in the reaction mixture at the onset of the execution of step b)—to be discussed below—may vary between wide limits; preferably, the said amount is at least 1, 2, 3 or 5 mol. %; more preferably at least 7, 10 or 15 mol. %. The said amount of O₂ and/or another oxygen-containing compound is preferably at most 60, 50 or 40 mol. %, more preferably at most 40 or 25 mol %. The percentages as given here for the amount of O₂ and/or another oxygen-containing compound are molar percentages and relate to the reaction mixture as a whole on the onset of execution of step b).

In view of the high temperatures in subsequent step b) of the process according to the invention and the need to bring the reaction mixture in contact with a solid catalyst at space velocities that may be relatively high, the reaction mixture should be in the gaseous state—or at least in the supercritical state. If this is not already the case when the reaction mixture is initially formed, then a gasification step should be executed during step a) or subsequent to it—but prior to step b).

In step b) of the process according to the invention, the reaction mixture is brought into contact with a catalyst. The catalyst contains a metal M₁ on a support. Within the context of the present invention, the term ‘metal M₁’ or ‘M₁’ is understood to mean a metal compound itself, a metal oxide or a mixture of metal compounds and/or metal oxides. According to the invention, M₁ is a transition metal from group 3, 4, 5, 6, 7, 8, 9, 10, 11 and 12 of the IUPAC Periodic Table of Elements, or a mixture thereof. A current Internet reference for the IUPAC Periodic Table of Elements is www.iupac.org/reports/periodic_table/; the version as used here is dated 3 Oct. 2005. Preferably, M₁ is selected from group 8,9,10 and 11 consisting of Fe, Ru, Os, Co, Rh, Ir, Ni, Pd, Pt, Cu, Ag and Au their respective oxides, and mixtures thereof. More preferably M₁ is selected from the group consisting of Ru, Rh and Cu their respective oxides and mixtures thereof.

Metal M₁ is present on or in a support. As support, preferred use is made of heat resistant inorganic compounds. Within the context of the present invention, the term ‘support’ is understood to mean one heat resistant inorganic compound or a mixture of two or more heat resistant inorganic compounds. Examples of such compounds are alumina, silicon carbide or other carbon-containing supports, silicon oxide, titanium oxide, silica magnesia, magnesium oxide, diatomaceous earth, prumice, zirconium oxide, cerium oxide, calcium sulphate, titanium phosphate, silicon phosphate and their mixtures. Among others, magnesium oxide is particularly preferable. When more than one metal M₁ is present in the catalyst, the different metals can be present on or in the same support or on or in different supports. The amount of the active components, i.e. components showing catalytic activity, to the total weight of the catalyst varies, depending a.o. upon the used support, method of preparing the catalyst, and atom ratio of the active components, but is generally at least 0.1, 0.5, 1, 2 or 5 wt. % and preferably at most 99, 95, 90, 80 or 70 wt. %.

The catalyst to be used in the process according to the invention may be prepared by methods known as such to the skilled person. An example of such a method is the vapour-phase decomposition of a salt of M₁ in the presence of the support, followed by grinding and a heat treatment. In preparing the catalyst to be used in the process according to the invention, it is preferably ensured that M₁ is primarily present on the support rather than in it: M₁ should preferably not be present in the form of a homogeneous mixture with the support, but rather attached to the surface of the support, for example deposited on the surface of particles that consist of the support material.

The catalyst as used in the process according to the invention can have various shapes, such as for example small particles or granules or wires or gauzes. If the catalyst comprises or consists essentially of particles—either as such or in agglomerated or sintered form—then it is preferred that the said particles are in size between 100 nm and 5 mm. The term size is defined herein as the average value of the largest and smallest dimension of a particle.

Since the catalyst is essentially in the solid phase and the reaction mixture is essentially in the gaseous or supercritical phase, it follows that step b) according to the invention falls into the category of heterogeneous catalytic reactions.

In step b) of the process according to the invention the reaction mixture, optionally combined with an additional stream containing H₂— and/or O₂, is brought into contact with the catalyst. This is being done under certain conditions of temperature and space velocity. The temperature at which step b) is to be executed lies between 200° C. and 800° C. The temperature should be at least 200° C. or 250° C., preferably 300, 400; more preferably 500 or 525° C.; this has the advantage that an acceptable speed of reaction can be achieved. The temperature should be at most 800° C. or 750° C., preferably at most 700° C. or 650° C., most preferably at most 600° C. or 575° C.; this has the advantage that undesirable side-reactions, e.g. leading to destruction or total oxidation of the raw materials, are reduced or even essentially avoided. The temperature as required for executing step b) of the invention may be reached through heating measures that are as such known, such as via heat exchangers. In a preferred embodiment, however, the heating of the reaction mixture and/or the catalyst is not achieved solely or even partly through microwave irradiation or corona discharge, as these methods may have the disadvantage that undesirable side reactions may occur.

Step b) according to the invention may be carried out in a wide range of pressures but preferably between 0.1 or 0.15 MPa and 30 MPa, more preferably between 1 or 2 MPa and 25 or 20 MPa.

Step b) of the process according to the invention should be carried out at a space velocity lying between 102 and 106 millilitre of reaction mixture per gram of catalyst per hour (ml/(g.h)). Without committing to scientific explanation, it is thought that a space velocity of at least 102 ml/(g.h), preferably at least 3.102, 10³ or 3.103 ml/(g.h) is needed so as to minimize the occurrence of undesired side-reactions such as total oxidation of raw materials; also, it is thought that a space velocity below 10⁶ ml/(g.h), preferably below 3.105 ml/(g.h) or 10⁵ ml/(g.h) should be chosen so as to ensure that the nitrogen-containing compound formation can indeed take place.

It may be beneficial to execute step b) in multiple subsequent stages; an example hereof is the execution of step b) in two consecutive stages b1) and b2). Although in both of the stages b1) and b2) the reaction mixture is brought into contact with a catalyst as defined herein, the characteristic of executing step b) in multiple stages is that process features such as temperature and pressure but also other features like the composition of the catalyst may be varied. An advantage of executing step b) in multiple stages is that if the formation of the nitrogen-containing compound proceeds more favourably through one or more intermediate reactions then the optimal conditions such as temperature, pressure and catalyst composition for each of the intermediate reactions may be selected individually in the respective stages of step b). It is preferred that at least one of the following features: temperature, pressure, and catalyst composition is different in b2) compared to b1). Within the context of the present invention, the term different should be interpreted as meaning a difference of:
at least 25° C., preferably between 50° C. and 200° C. if the temperature is chosen as differentiating feature;
at least 10% of the lowest pressure value in b1) or b2) if the pressure is chosen as differentiating feature;
the type of M₁ and/or the type of support and/or at least 30 wt. % in the amount of M₁, calculated on the amount of M₁, if the catalyst composition is chosen as differentiating feature.
Sub-stages b1) and b2) may be executed in one reactor, but it may be for practical reasons beneficial to execute them in two separate consecutive reactors.

In a further preferred embodiment, step b) is executed in three or even four or more subsequent stages b1), b2), b3) and possibly b4). Also here it may be beneficial or even necessary that the features temperature, pressure and/or catalyst composition constitute a differentiating feature between the stages of step b). In one preferred embodiment, at least temperature is chosen as a feature that is different between the stages of step b). For example, stage b1) may be executed at a temperature lying between 375° C. and 425° C., b2) at a temperature lying between 500° C. and 625° C., and b3) at a temperature lying between 325° C. and 475° C. In another embodiment step b) is executed in two stages b1) and b2) having temperatures lying between 375° C. and 425° C. and between 525° C. and 575° C., respectively.

In the multi-stage embodiments of step b) of the invention, it may be preferable or even necessary to separate off a part of the reaction mixture in between the stages, as a side stream. Such separating off of a side stream may be non-specific or it may be selective by means of a selective separating technology such as distillation. An example of a compound that may be the target of being separated off selectively is hydrogen; another example is ammonia.

After having been separated off, it may be beneficial to recycle the side stream to a previous stage within step b), e.g. in case the formation of the nitrogen-containing compound or intermediate compounds is incomplete.

In the multi-stage embodiments of step b) of the invention, it may be beneficial or even necessary to add one of the starting compounds or one or more additional compounds to the reaction mixture between stages or in a stage. It is thus possible to bring in step a) not all the starting compounds together, but to add one starting compound at a later stage during step b). Examples of such compounds are ammonia and carbonmonoxide.

As a result of step b) according to the invention, a nitrogen-containing compound or mixture of nitrogen-containing compound is formed. Essential in nitrogen-containing compounds is that a carbon-nitrogen bond is present; it is a major objective of step b) according to the invention that such carbon-nitrogen chemical bonds are being formed. A characteristic hereby is that neither the carbon nor the nitrogen as comprised in the nitrogen-containing compound originate to any significant extent from the catalyst but, rather, originate essentially from the raw materials only.

In a preferred embodiment of the invention, step b) is followed by a step c) in which a portion of the reaction mixture is separated off therefrom. The separated portion is herein defined as the recycle stream. The recycle stream is then, as disclosed above, combined with the raw materials—i.e. N₂ and a carbon- and hydrogen-containing compound or a carbon-containing compound and H₂—so as to become part of the reaction mixture that enters into step b). The portion of the reaction mixture that is separated off to become the recycle stream may vary within wide limits, preferably between 1 vol. % and 99 vol. % of the reaction mixture as it enters step b). More preferably, the portion that is separated off is between 5 vol. % and 50 vol. %, in particular between 10 vol. % and 25 vol. % of the reaction mixture as it enters step b). Preferably, no compounds are added to the recycle stream, in particular no ammonia. If step b) is executed in multiple stages such as in two stages b1) and b2), then it is in an embodiment of the invention preferred to feed at least 50 wt. % of the recycle stream to stage b1). In an alternative embodiment, however, it is preferred to feed at least 50 wt. % of the recycle stream to stage b2).

Subsequent to step b) or c) of the invention, the nitrogen-containing compound can be isolated from the reaction mixture if so desired. This may be achieved by methods as such known to the man skilled in the art, such as condensation, bubble-extraction, etc.

The process according to the invention will be further illustrated by means of the following examples, without being limited thereto.

### EXAMPLE 1

A Ru/MgO catalyst was prepared by vapour-phase decomposition of a ruthenium salt (triruthenium dodecarbonyl) in the presence of MgO powder. 1 gram of MgO (99.99% purity) and 0.111 gram of triruthenium dodecarbonyl were mixed thoroughly and ground for 30 minutes. The mixture thus prepared was treated under vacuum at 450° C. for 5 hours.

A micro reactor was filled with 32 mg of the Ru/MgO catalyst, whereby the catalyst was diluted in 150 mg silica to ensure plug flow conditions. A He/O₂ mixture was fed to the reactor; the temperature in the reactor was raised by 5° C. /min to 450° C. and kept there; after 30 minutes at 450° C., the feed was switched to a mixture of He and H₂ for 2 hours, after which step a) and b) were executed.

A flow consisting of a mixture of N₂ and H₂ was fed to the reactor at a temperature of 400° C. Into the feed, CH₄ (methane) was pulsed in amounts of 4 μmole per pulse. After each pulse, the Infrared (IR) spectrum was recorded. In the first part of this Example, the N₂ was ¹⁴N₂; in the second part of this Example, the N₂ feed was switched from ¹⁴N₂ to ¹⁵N₂.

During the first part of the Example an IR peak at 2194 cm⁻¹ was determined; this peak is assigned to (CH₂C¹⁴N)⁻. In the second part of the example, i.e. after the N₂ feed had been switched from ¹⁴N₂ to ¹⁵N₂, the said peak shifted to 2174 cm⁻¹; this is a peak associated with (CH₂C¹⁵N)⁻.

The pulse method of feeding the carbon-containing compound has the disadvantage that it is not possible to identify precisely the space velocity of the said compound during the reaction. Nevertheless, the information as derivable from using labelled nitrogen in Example 1 yielded evidence that the raw material N₂ was indeed consumed for the formation of a carbon-nitrogen chemical bond in a nitrogen-containing compound having overall formula CₓH_{y}N.

### EXAMPLE 2

A Ru/MgO catalyst was prepared as in Example 1 by vapour-phase decomposition of a ruthenium salt (triruthenium dodecarbonyl) in the presence of MgO powder. 1 gram of MgO (99.99% purity) and 0.111 gram of triruthenium dodecarbonyl were mixed thoroughly and ground for 30 minutes. The mixture thus prepared was treated under vacuum at 450° C. for 5 hours.

A micro reactor was filled with 48 mg of the Ru/MgO catalyst, whereby the catalyst was diluted in silica to ensure plug flow conditions. A He/O₂ mixture was fed to the reactor; the temperature in the reactor was raised by 5° C. /min to 450° C. and kept there; after 30 minutes at 450° C., the feed was switched to a mixture of He and H₂ for 2 hours, after which step a) and b) were executed. The temperature in the reactor was raised to 600° C. and gas was led through the reactor with a flow rate of 80 ml/min; the gas flow consisted of 4 ml/min CH₄, 10 ml/min N₂, 30 ml/min H₂, and 36 ml/min He. The space velocity over the catalyst was 100,000 ml/(g.h). The gas that exited the reactor was analysed; of the amount of carbon as fed to the reactor, 1.24 ppm was found to have reacted into dimethylamine, 0.05 ppm into pyridine and 0.26 ppm into melamine.

Example 2 clearly demonstrates that the process according to the invention leads to the formation of a nitrogen-containing compound.

### EXAMPLE 3

A Ru/MgO catalyst was prepared as in Example 1 by vapour-phase decomposition of a ruthenium salt (triruthenium dodecarbonyl) in the presence of MgO powder. 1 gram of MgO (99.99% purity) and 0.111 gram of triruthenium dodecarbonyl treated under vacuum at 450° C. for 5 hours.

A micro reactor was filled with 50 mg of the Ru/MgO catalyst, whereby the catalyst was diluted in silica to ensure plug flow conditions. A He/O₂ mixture was fed to the reactor; the temperature in the reactor was raised by 5° C. /min to 450° C. and kept there; after 30 minutes at 450° C., the feed was switched to a mixture of He and H₂ for 2 hours, after which step a) and b) were executed. Gas was led through the reactor with a flow rate of 11 ml/min; the gas flow consisted of 4 ml/min CO, 2 ml/min N₂, and 5 ml/min H₂. The space velocity over the catalyst was 13,200 ml/(g.h). Step b) was executed at atmospheric pressure. The gas that exited the reactor subsequent to step b) was analysed by means of on-line mass spectrometry (MS). Of the amount of carbon as fed to the reactor in the form of CO, 1308 ppm was found to have reacted into compounds that gave a signal at mass 27, which is in this experimental constellation evidence to the forming of HCN or compounds containing the HCN building block.

Also Example 3 clearly demonstrates that the process according to the invention leads to the formation of a nitrogen-containing compound.

### EXAMPLES 4-7

Example 3 was repeated, except that the temperature in the reactor was not 450° C. but was set to 400, 500, 550 and 600° C. The yield in compounds that gave an MS signal at mass 27—based upon the amount of carbon as fed to the reactor in the form of CO—was as given in the table below.

| | | ppm of |
|---|---|---|
| | Temperature | C-source |
| Example | (° C.) | converted |
| 4 | 400 | 1318 |
| 5 | 500 | 482 |
| 6 | 550 | 84 |
| 7 | 600 | 49 |

### EXAMPLES 8-10

Example 3 was repeated, with however the following differences:
The temperature in the reactor was set to 400, 500 or 600° C.
The gas as fed to the reactor had a flow rate of 12 ml/min and consisted of 2 ml/min CH₄, 1 ml/min O₂, 1 ml/min N₂ and 8 ml/min He
The space velocity over the catalyst was 14,400 ml/(g.h)

The yield in compounds that gave an MS signal at mass 27—based upon the amount of carbon as fed to the reactor in the form of CH₄—was as given in the table below.

| | | ppm of |
|---|---|---|
| | Temperature | C-source |
| Example | (° C.) | converted |
| 8 | 400 | 564 |
| 9 | 500 | 473 |
| 10 | 600 | 351 |

1. Process for the preparation of a nitrogen-containing compound, comprising the steps of:
a) bringing N₂, optionally NH₃ and optionally a recycle stream together with a carbon- and hydrogen-containing compound or a carbon-containing compound and H₂ to form a reaction mixture, whereby the ammonia in the reaction mixture, if present, originates for at least 30 wt. % from the recycle stream;
b) bringing the reaction mixture in contact with a catalyst at a temperature lying between 200° C. and 800° C. and at a space velocity lying between 10² and 10⁶ ml/(g.h), said catalyst containing a metal M₁ on a support, M₁ being chosen from the group consisting of metals in group 3, 4, 5, 6, 7, 8, 9, 10, 11 and 12 of the IUPAC Periodic Table of Elements or mixtures thereof, whereby the nitrogen-containing compound is formed;
c) optionally separating, subsequent to step b), a portion of between 1 and 99 vol. % off from the reaction mixture, said portion being the recycle stream.

2. Process according to claim 1, wherein in step a) and/or in step b) H₂ and/or carbonmonoxide is added to the reaction mixture.

3. Process according to claim 1, wherein in step a) and/or in step b) O₂ and/or an oxygen-containing compound is added to the reaction mixture.

4. Process according to claim 1, wherein in step a) and/or step b) H₂O is added to the reaction mixture.

5. Process according to claim 1, wherein step b) is executed at a pressure lying between 0.15 MPa and 25 MPa.

6. Process according to claim 1, wherein step b) is executed at a temperature lying between 350° C. and 600° C.

7. Process according to claim 1, wherein the catalyst further contains a second metal M₁, M₁ being chosen from the group consisting of metals in group 3, 4, 5, 6, 7, 8, 9, 10, 11 and 12 of the IUPAC Periodic Table of Elements or mixtures thereof.

8. Process according to claim 1, wherein M₁ contains Ru and the support contains MgO.

9. Process according to claim 1, wherein the catalyst comprises particles, whereby the size of the said particles lies between 100 nm and 5 mm.

10. Process according to claim 1, wherein the carbon- and hydrogen-containing compound or carbon-containing compound consists essentially of CH₄.

11. Process according to claim 1, wherein step b) is executed in three subsequent stages b1), b2) and b3), whereby in the stages b1), b2) and b3) the reaction mixture is brought into contact with a catalyst, and whereby at least one of the following features:
temperature,
pressure,
catalyst composition, is different in b2) compared to b1) and b3) and is different in b3) compared to b1) and b2) the difference being:
at least 25° C. if the temperature is chosen as differentiating feature;
at least 10% of the lowest pressure value in b1) or b2) if the pressure is chosen as differentiating feature;
the type of M₁ and/or the type of support and/or at least 30 wt. % in the amount of M₁, calculated on the amount of M₁, if the catalyst composition is chosen as differentiating feature.

12. Process according to claim 11, wherein the recycle stream is for at least 25 wt. % fed to stage b1).
